(19) United States
(12) Patent Application Publication
(10) Pub. No.: US 2007/0161103 A1
(43) Pub. Date: Jul. 12, 2007

(54) Device and method for the visual analysis of test strips

(75) Inventors: Andreas Buchmann, Dietikon (CH); Stefan Jenni, Urdorf (CH); Alexander Rietsch, Dietikon (CH); Michael Sidler, Forch (CH); Bruno Oesch, Stilli (CH)

Correspondence Address: RANKIN, HILL, PORTER & CLARK LLP, 4080 ERIE STREET, WILLOUGHBY, OH, 44094-7836, US

(73) Assignee: PRIONICS AG, Schlieren (CH)

(21) Appl. No.: 10/597,687

(22) PCT Filed: Feb. 18, 2005
(86) PCT No.: PCT/EP05/01691

§ 371 Date: Oct. 4, 2006

(30) Foreign Application Priority Data
Feb. 19, 2004 (DE) 10 2004 008 539.0

(51) Int. Cl. C12M 1/34

(52) U.S. Cl. 435/287.2; 382/128

(57) ABSTRACT

A device for the visual analysis of test strips, each having at least one delimited area in which, after contact with a sample to be examined, a visually detectable signal can be generated, with the device having a positioning device including a seat for at least one test strip or at least one test strip unit consisting of a plurality of test strips combined in a defined spatial arrangement, the device further including an image generating device which graphically records at least one of the delimited areas and transmits the recording result to an image analysis device, with the image analysis device qualitatively and/or quantitatively analyzing the visually detectable signals for each test strip.

### BACKGROUND OF THE INVENTION

1. Field of the Invention

The present invention relates to a device for the visual analysis of test strips as well as to a method for the visual analysis of test strips.

2. Description of Related Art

Test strips are frequently used, for example, in clinical diagnostics to diagnose easily recordable physiological parameters, metabolites or pathogens.

Test strips are used in the detection of numerous different analytes in liquid or homogenized samples. As a rule, at least one delimited area is provided on the test strips in which a detection reagent is immobilized for the specific analyte. Such test strips are used, for example, to detect glucose in urine, or for blood glucose analysis, or for detecting prionic proteins in liquid or liquefied or, respectively, homogenized samples. The last mentioned test strip is known from DE 10147012 by the applicant of the present invention.

As a rule, the test strips are designed such that, in the delimited area, following contact with the sample to be analyzed, a visually detectable signal can be generated which can then be detected by the investigating person through visual inspection or by means of an image analysis device.

The visually detectable signal can be, for example, a color change, a lighter or darker shade, or a change in fluorescence.

It is furthermore known from DE 10147012 that a plurality of test strips can be combined a comb-like test strip unit, with the test strips being combined in a defined geometric arrangement to each other so that their lower sections can be simultaneously inserted into the sample tubes arranged in rows in a micro-titer plate.

Furthermore, it is known from DE 10147012 to combine a plurality of such test strip units by means of a connecting device so that the lower sections of the test strips of the individual test strip units can be simultaneously inserted into the sample tubes arranged in rows in a micro-titer plate.

It is, thus, possible to simultaneously examine a great number of samples from different subjects by means of the test strips and to, thus, substantially increase the throughput rate in routine diagnostics.

When using a plurality of test strip units, there is a problem of allocating the individual test strips or, respectively, the analysis results of the visually detectable signals on the individual test strips to the appropriate subjects. Confusion can happen quickly in this situation.

Moreover, it is laborious to register the visually detectable signals on each test strip by means of visual inspection and allocate them to the appropriate subject or, respectively, have the individual test strips or test strip units analyzed by an image analysis device.

### BRIEF SUMMARY OF THE INVENTION

It is the objective of this invention to provide a device for the visual analysis of test strips which will allow analysis, at a high rate, of the visually detectable signals on test strips possibly combined to a test strip unit and to allocate them with high reliability to the appropriate subject.

In accordance with the invention, a device for the visual analysis of test strips is provided, with the test strips each having at least one delimited area in which a visually detectable signal can be generated after contact with a sample to be examined.

The device comprises a positioning device which has a seat for at least one test strip and an image generating device which graphically records at least one of the delimited areas of a test strip arranged in the positioning device and/or a test strip unit, and which transfers the recording result to an image analysis device, with the image analysis device qualitatively and/or quantitatively analyzing the visually detectable signals for every test strip.

It is preferably provided that the positioning device comprises seats for a plurality of test strips and/or test strip units.

As a rule, the visually detectable signal is correlated with a chemical or immunological reaction which can take place in the delimited area or areas after contact with a sample to be examined. For this purpose, reagents, enzymes or antibodies are immobilized in the delimited areas. The visually detectable signal can consist, for example, of a color change, a lighter or darker shade, or a change in fluorescence. In the event that a reaction is negative because, for example, the metabolite to be detected is not present in the sample to be analyzed, the visually detectable signal can also be the absence of a signal.

For the qualitative analysis, it is sufficient that the image analysis device checks whether or not there is a visually detectable signal in the delimited areas. In contrast, for the quantitative analysis, it is first necessary that there is, in terms of the chemical or immunological reaction on which the signal is based, a mathematical relationship between the concentration of the physiological parameter or analyte to be detected and the strength or, respectively, the change of the visually detectable signal. This can be any mathematical relationship, such as e.g. a linear or an exponential relationship. If these prerequisites are given, a quantitative analysis of the visual signal will also be possible. This can be done by densitometric, colorimetric or fluorometric measurement of the delimited area or areas.

The use of individual test strips in routine diagnostics is very elaborate and inefficient. Thus, to increase the throughput rate in routine diagnostics, a plurality of test strips can be combined into a test strip unit such that the lower sections of the test strips of the individual test strip units can be inserted, for example, simultaneously into the sample tubes arranged in rows in an integrated sample tube system. These integrated sample tube systems can be, for example, commercially available micro-titer plates or rack systems for micro reaction tubes.

In a preferred embodiment of the device according to the invention, it is accordingly provided that a plurality of test strips is each combined in a defined spatial arrangement to form a test strip unit, and that the positioning device comprises a seat for at least one test strip unit. It is preferably provided that the positioning device comprises seats for a plurality of test strip units. The image analysis device also analyzes the visually detectable signals qualitatively and/or quantitatively for every test strip.

The test strip units applicable according to the invention can consist of individual non-contiguous test strips which are connected with each other by means of a connecting arrangement, for example by clamping or gluing, extending transversely to the test strips. In the same manner, the test strip units with the individual test strips can also be formed as one single piece, for example, from one cut. Yet, even individual test strips can be used which are not combined in a test strip unit.

A preferred embodiment of the device according to the invention provides that the image generating device is a scanner. This can be a commercially available flatbed scanner of the size A4. The image generating device can just as well be a digital camera which is provided on a tripod or on a robotic arm above the positioning device. But the use of a CCD element or another suitable image generating device is possible as well.

A specially preferred embodiment of the device according to the invention provides that the positioning device is designed such that it can be arranged in the scanner preferably provided. When a flatbed scanner is used, it can be provided such that the positioning device is placed on the flatbed scanner.

Yet, the image generating device can also be a portable device which, for example, graphically records only one test strip or one test strip unit. Such a device would be particularly suitable for individual case diagnostics or the diagnostics for few subjects. Such an embodiment would have great advantages especially in field applications where no PC is available. In this case, the positioning device could be, for example, firmly installed in the image generating device, for example, as a cutout, hidden under a flap, for a test strip unit. But the positioning device could also consist, if necessary, of a pull-in device which pulls a single test strip in through a slot. Still other embodiments are conceivable, however.

Basically, any device is suitable as a positioning device in which at least one test strip or one test strip unit can be arranged, and which in turn can be arranged to or, respectively, in or on an image generating device such that the image generating device can record the delimited area or areas of test strips arranged in the positioning device.

It is especially preferably provided that the positioning device and the image generating device are designed such that the positioning device can be arranged in a reproducible manner in a defined arrangement on or, respectively, in the image generating device. In this manner, it will be ensured that the recording result generated by the image generating device will always comprise the same area or areas of the positioning device.

This can be done, for example, by providing cutouts in the positioning device into which pins will engage when it is placed on a flatbed scanner, to thus secure the positioning device on the flatbed scanner.

The positioning device preferably consists of a frame, and the seats for the test strip units preferably consist of cutouts in this frame. The frame can consist of a plastic such as PVC.

It can be provided that the test strip units or, respectively, individual test strips, if these are used instead of the test strip units, are inserted into cutouts which are appropriately adjusted to their size and shape. It is especially preferably provided that the cutouts and the test strip units or, respectively, the test strips are designed such that the test strip units or the test strips, upon arrangement in the cutouts, can latch into them. For this purpose, it can be provided that the test strip units have edge reinforcements provided with a lateral groove into which, upon arrangement in the seats of the positioning device, an elastic lip engages which is arranged there. However, yet other embodiments are conceivable which enable latching of the test strip units or the test strips into the cutouts.

Moreover, it can be provided that the cutouts and the test strip units are designed such that a given test strip unit can each be arranged in a positive fit only in a defined seat of the positioning device. For this purpose, the edges of the seats can comprise protruding elements which, according to the lock and key principle, engage into the appropriate cutouts in the edge reinforcements of the test strip units. This embodiment can also be transferred to the use of individual test strips.

A preferred embodiment of the device according to the invention provides that the positioning device comprises at least two visually detectable position markers.

Another embodiment of the device according to the invention provides that the image analysis device comprises a computer with image processing software.

It is especially preferably provided that the image generating device graphically records the position markers and transmits the recording result to the image analysis device which can localize the test strip units, the test strips, and/or the delimited areas by means of the position markers and thus reproducibly detect them.

For this purpose, it is required that the position markers are arranged at known distances and angles to the individual seats for the test strip units or test strips, and that, furthermore, the relative positions of the test strip units, and/or the test strips, as well as of the delimited areas to each other are known.

Furthermore, a preferred embodiment of the device according to the invention provides that the test strip units and/or the test strips comprise visually detectable, individualizing markings.

These may include information on the manufacturing batch, the best before date of the test strip unit or of the test strips, information on the present test or the analysis protocol to be used, or also user information such as the identity of the subjects, the date of the test, etc. Preferably, the individualizing markings are bar codes such as can be easily read by a bar code reader, but also by a commercially available scanner with a suitable image analysis device.

Usually, a plurality of test strip units or test strips are combined in lots and delivered to the end user. Within the scope of the invention, it can be furthermore provided that a calibration slip is enclosed with each lot, on which relevant data of the lot or of the test strip units contained therein are listed in machine readable form, for example, in the form of a two-dimensional bar code. Prior to the first measurement of, for example test strip units of a new lot, this slip will be arranged in the place of the positioning device in the device according to the invention and will be read in with the image generating device. It may be provided that reading in the calibration slip is absolutely imperative, i.e. measurements of the test strip units of a new lot can only be taken afterwards.

A specially preferred embodiment of the device, according to the invention, provides that the image generating device graphically records the individualizing markings and transmits the recording result to the image analysis device which identifies the individual test strip units and/or the test strips based on the individualizing markings and uses the data codified in the markings for the analysis, if necessary. For this reason as well, it is necessary that the relative positions of the individualizing markings as well as of the test strip units and/or the test strips as well as of the delimited areas to each other are known.

Furthermore, in an embodiment of the invention, the test strip units and/or the test strips also comprise at least two visually detectable position markers. This characteristic is a prerequisite for a specially preferred embodiment of the invention according to which the image generating device graphically records the position markers on the test strip units or the test strips and transfers the recording result to the image analysis device which can localize the test strip units, the test strips and/or the delimited areas by means of the position markers and can thus detect them reproducibly. Here again, it is required that the position markers are arranged at known distances and angles to the individual test strips so that the relative positions of the position markers, the test strip units and/or the test strips, as well as of the delimited areas to each other are known.

As already described, it is furthermore provided that the image analysis device qualitatively and/or quantitatively analyzes the visually detectable signals for every test strip. The quantitative analysis can here be done through densitometric, calorimetric or fluorometric measurements of the delimited area or areas.

The precision of the quantitative analysis may here be impaired, for example, due to fluctuations in the brightness of the scanner lamp or due to fluctuations in the brightness of the test strip material. To be able to compare the results of different quantitative analyses with each other, the image analysis device must accordingly be calibrated at regular intervals, for example, prior to every measurement, or the device must calibrate itself.

Another specially preferred embodiment of the device according to the invention provides that the positioning device comprises a visually detectable gray scale and/or a color scale. A gray scale or a color scale can be applied by the manufacturer with high reproducibility on the positioning device. If necessary, it can be designed such that it can be replaced at regular intervals to prevent bleaching, for example. To this end, the gray scale or the color scale can be designed in the form of a sticker.

This characteristic is a prerequisite for a specially preferred embodiment of the invention according to which the image generating device graphically records the gray scale and/or the color scale and transmits the recording result to the image analysis device which uses it for the calibration and for the analysis of the visually detectable signals on each test strip. As described, the analysis can be done by densitometric, calorimetric or fluorometric measurement of the delimited area or areas.

Another preferred embodiment of the device according to the invention provides that the individual test strips are arranged in parallel to each other and spaced apart from each other such that their lower sections can be simultaneously inserted into neighboring sample tubes of a tube row of an integrated sample tube system. These integrated sample tube systems can be commercially available micro-titer plates or rack systems for micro reaction tubes.

This characteristic facilitates the use of the test strip units with micro-titer plates and enables a great number of samples from different subjects to be simultaneous analyzed and the throughput rate in routine diagnostics to be thus substantially increased.

It is here especially preferably provided to design the test strip unit such that a plurality of test strip units are simultaneously insertable with their lower sections into the different tube rows of the integrated sample tube system.

Commercially available micro-titer plates with 96 sample tubes comprise twelve rows with eight sample tubes each. The sample tubes have a diameter of about 6 mm, and the individual sample tubes are at a distance of about 2 mm from each other. Accordingly, it is preferably provided that the test strip units comprise eight test strips each whose width and distances from each other are in accordance with the indicated dimensions. Other micro-titer plate formats comprise 24 or 48 rows with 16 or 32 sample tubes each. It is furthermore provided that the test strip units can also comprise 16 or 32 test strips, with their width and distances from each other also corresponding to the diameters and distances of the pertinent sample tubes.

The positioning device is preferably designed such that it can accept as many test strip units as are insertable in the different tube rows of an integrated sample tube system. If the integrated sample tube system is, for example, a micro-titer plate of a format of eight by twelve, test strip units with eight test strips would preferably be used. The positioning device would accordingly comprise twelve seats for test strip units.

Another preferred embodiment of the device, according to the invention, provides that the image analysis device performs a plausibility check by means of which it checks whether a test strip unit or, respectively, a test strip is provided in all seats of the positioning device, whether the individual test strip units or, respectively, test strips are arranged in a desired sequence in the seats of the positioning device, whether the test strip units or, respectively, the test strips come from the same manufacturing batch, and/or whether the best before date of the test strip units or, respectively, the test strips has already been reached.

The present invention is not restricted to the device as such but also concerns suitable, especially separately handleable positioning devices for a device according to any one of the preceding claims.

Suitable positioning devices form a surface which is graphically recordable by an image generating device and comprise at least one seat for a test strip unit or a test strip. Preferably, however, the positioning device comprises seats for a plurality of test strip units or test strips.

A preferred embodiment of the positioning device according to the invention provides that the positioning device consists of a frame, and the seats for the test strip units or the test strips consist of cutouts in the frame. Especially preferably, the cut-outs in the frame as well as the test strip units or the test strips are designed such that, when arranged in the cutouts, they can engage into them. However, it can also be provided that the test strip units or, respectively, the test strips are simply placed into the cutouts.

Another preferred embodiment of the positioning device according to the invention provides that the positioning device comprises at least two visually detectable position markers. Especially preferably, the positioning device according to the invention comprises a gray scale and/or a color scale.

The image analysis device provided in the device according to the invention preferably comprises software for the visual analysis of test strips. The test strips respectively comprise at least one delimited area in which, after contact with a sample to be examined, a visually detectable signal can be generated; and, on the basis of the recording result of at least the delimited areas transferred by an image generating device, the software determines the position of the individual test strips and/or the delimited areas, identifies the individual test strip units, test strips, and/or the delimited areas, and qualitatively and/or quantitatively analyzes the visually detectable signals for every test strip.

A preferred embodiment provides that a plurality of test strips each are connected in a defined spatial arrangement to a test strip unit, and the software determines the positions of the test strip units, identifies the individual test strip units, and qualitatively and/or quantitatively analyzes the visually detectable signals for every test strip.

Another preferred embodiment provides that the software localizes and thus reproducibly detects the test strip units, the test strips, and/or the delimited areas by means of position markers, arranged on the positioning device, the test strip units and/or the test strips, which are graphically recorded by the image generating device.

A specially preferred embodiment provides that the software identifies the individual test strip units and/or the individual test strips based on individualizing markings arranged on the test strip units and/or the test strips and graphically recorded by the image generating device, and that the software uses the data codified in the markings for the analysis, if necessary.

It is preferably furthermore provided that the software analyzes the visually detectable signals on the test strips by means of a gray scale and/or color scale graphically recorded by means of an image generating device. The quantitative analysis can be done e.g. by densitometric, calorimetric or fluorometric measurement of the delimited area or areas.

It is here preferably provided that the software performs a plausibility check by means of which it is checked, for example, whether a test strip unit or a test strip is provided in all seats of the positioning device, whether the individual test strip units or test strips are arranged in a desired sequence in the seats of the positioning device, whether the test strip units or the test strips are from the same manufacturing batch and/or whether the best before date of the test strip units or test strips has already been reached.

The present invention, furthermore, covers a method for the visual analysis of test strips in which the test strips comprise at least one delimited area in which, after contact with a sample to be examined, a visually detectable signal can be generated. At least one test strip will be arranged in a seat of a positioning device which can be arranged on an image generating device, and by means of the image generating device, at least one of the delimited areas is graphically recorded. Preferably, a plurality of test strips is arranged in seats of the positioning device.

The recording result will be transmitted to an image analysis device which determines and identifies the positions of the individual test strips and/or the delimited areas, and qualitatively and/or quantitatively analyzes the visually detectable signal for every test strip.

A preferred embodiment of the method according to the invention provides that a plurality of test strips each is combined in a defined spatial arrangement to a test strip unit and that at least one test strip unit is arranged in a seat of a positioning device. Preferably, a plurality of test strip units is arranged in seats of the positioning device.

The image analysis device determines the positions of the individual test strip unit or units, identifies them and qualitatively and/or quantitatively analyzes the visually detectable signal for each test strip.

A specially preferred embodiment of the method according to the invention provides that, by means of the image generating device, position markers arranged on the positioning device, the test strip units or the test strips are graphically recorded, and the recording result is transmitted to the image analysis device which can localize and, thus, reproducibly detect the test strip units, the test strips and/or the delimited areas by means of the position markers.

Another preferred embodiment of the method according to the invention provides that the image generating device graphically records individualizing markings arranged on the test strip units or the test strips, and transmits the recording result to the image analysis device which identifies the individual test strip units and/or the individual test strips on the basis of the individualizing markings, and uses the data codified in the markings for the analysis, if necessary.

A specially preferred embodiment of the method according to the invention provides that the image generating device graphically records a gray scale and/or a color scale arranged on the positioning device and transmits the recording result to the image analysis device which uses it as a calibration scale for the analysis of visually detectable signals on every test strip. Here, the quantitative analysis can be done by densitometric, calorimetric or fluorometric measurement of the delimited area or areas.

Furthermore, the device according to the invention relates to usable test strip units comprising a plurality of test strips arranged with respect to each other in a defined spatial arrangement, with the test strips, each comprising at least one delimited area in which, after contact with a sample to be analyzed, a visually detectable signal will be generated. Here, a plurality of test strips is combined in a defined spatial arrangement to a test strip unit. The test strips comprise an absorbable cut in which at least one delimited area each is provided in which, after contact with a sample to be analyzed, a visually detectable signal can be generated. The absorbable cut consist of such a material. Nitrocellulose is especially preferably used for it; but any other material suitable for this purpose is also conceivable.

A specially preferred embodiment of this test strip unit provides that the absorbable cuts of the individual test strips are designed in one piece connected with each other. It is especially preferably provided here that the test strip unit comprises an edge reinforcement extending transversely to the test strips, arranged in absorption direction above the delimited areas. This edge reinforcement can consist of a plastic strip which is glued onto the test strip material. The edge reinforcement can facilitate the handling of the test strip units and in turn can bear the already described position markers and/or individualizing markings. Moreover, the edge reinforcement can be provided with a groove into which, upon arrangement in the seats of the positioning device, one or several elastic lips can engage which are arranged there. In this manner, the test strip units can latch into the seats of the positioning device.

Another embodiment of the test strip unit can also provide, however, that the test strip unit comprises individual, non-contiguous test strips which are connected with each other by means of a connecting device arranged in an absorption direction above the delimited areas and extending transversely to the test strips. In this case, an composite test strip unit is concerned. Here, the test strips can be glued or tacked into the connecting device, or held by it by means of clamping. Any other means of fastening is also conceivable. The connecting device can also function here at the same time as an edge reinforcement in the sense already described; thus, it can be designed such that it facilitates the handling of the test strip units, carries the already described position markers and/or individualizing markings, or enables latching into the cutouts of the positioning device.

It is furthermore preferably provided that the absorbable cut of the test strip is applied onto a stiff carrier material. This may be, for example, a plastic material. The carrier material facilitates the handling of the test strip units and prevents that the test strips will be deformed upon insertion into the sample tubes of the micro-titer plates.

Another preferred embodiment of the test strip unit, according to the invention, provides that the test strip unit is designed such that the individual test strips are arranged parallel to each other and spaced apart from each other such that its lower sections can be simultaneously inserted into adjacent sample tubes of a tube row of an integrated sample tube system. These integrated sample tube systems can be commercially available micro-titer plates or rack systems for micro reaction tubes.

It is especially, preferably provided that the test strip unit is designed such that a plurality of test strip units is simultaneously insertable into different tube rows of an integrated sample tube system.

Another preferred embodiment of the test strip unit according to the invention provides that the test strip unit comprises a waste pad arranged above the delimited areas which is used for the absorption of any excess liquid, if necessary. This waste pad can consist of pressed cellulose, for example.

Preliminary investigations by the applicant showed that there are difficulties in the production of test strip units in which the absorbable cuts of the individual test strips are designed in one piece connected with each other. This is especially due to the fact that the absorbable material used for the cuts is frequently very tough and, with standard stamping methods, can hardly be brought into the desired comb-like shape. This is especially true if the absorbable material is nitrocellulose.

Accordingly, another aspect of the invention relates to a stamping method for the production of a test strip unit in which the absorbable cuts of the individual test strips are designed as one piece connected with each other.

In this case, a blank for a test strip unit which consists of at least the material for the absorbable cut will be placed onto a stamping plate and at least the absorbable cut of the test strip unit will be stamped out. As mentioned, the blank consists of at least one layer of the absorbable material. However, it is also possible to use a multi-layer blank in which, for example, one layer of an absorbable material is applied on a stiff carrier material.

In the stamping method according to the invention, a stamping plate is used which has a negative profile in accordance with the cuts of the test strip unit, with the blades of the stamping tool employed having a falling profile which, in the stamping process, engages successively into the cutouts of the negative profile of the stamping plate.

Consequently, the areas to be removed from the blank are not actually stamped out but rather cut out during the stamping. The method allows, for the first time, the economical production of test strip units in large numbers in which the absorbable cuts of the individual test strips are designed in one piece and connected with each other.

The test strip units consisting of individual, non-contiguous test strips cannot be produced with the above stamping method. Here, a method for the production of a test strip unit is provided in which the test strip unit is produced from a plurality of individual test strips which are connected with each other by means of a connecting device extending transversely to the test strips. This method has the advantage, versus the before mentioned method, that the scrap of test strip material can be reduced. The individual test strips can be cut out in a conventional manner by means of a cutting machine and then combined to a test strip unit, as described. The test strips can be glued or tacked into the connecting device or held by it by means of clamping. However, any other means of fastening is also conceivable. The connecting device can also work at the same time as an edge reinforcement in the sense already described, thus, it can be designed such that it facilitates the handling of the test strip units, carries the already described position markers and/or individualizing markings, or enables latching into the cutouts of the positioning device.

### BRIEF DESCRIPTION OF THE DRAWINGS

In the drawings, the invention is presented diagrammatically and by way of example. It is shown in

FIG. 1 a perspective view of a test strip unit comprising a plurality of test strips;

FIG. 2 a perspective view of a positioning device with seats for test strip units;

FIG. 3 a perspective view of another test strip unit;

FIG. 4 a stamping tool for the production of a test strip unit according to the invention;

FIG. 5 a perspective view of a test strip unit with an edge reinforcement; and

FIG. 6 a perspective view of a test strip unit with a connecting device.

### DETAILED DESCRIPTION OF THE INVENTION

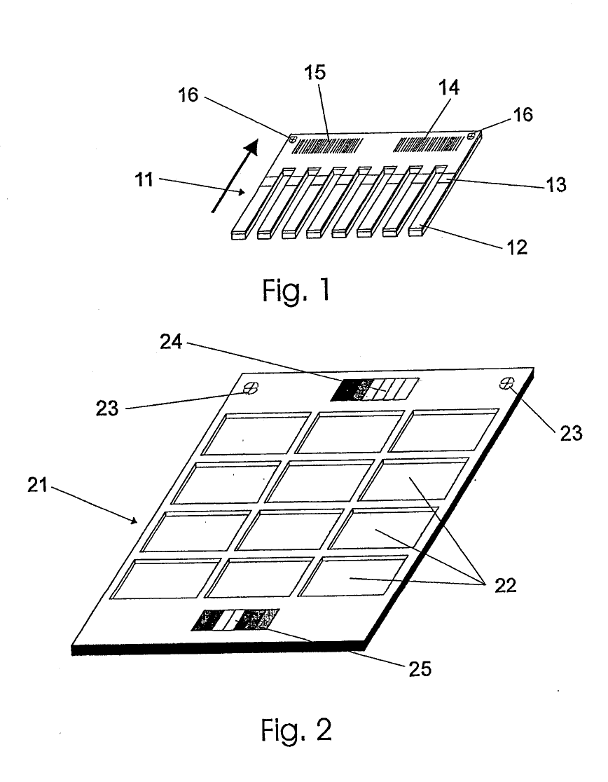

FIG. 1 shows a test strip unit 11, consisting of a plurality of test strips 12 each comprising a delimited area 13 in which, after contact with a sample to be examined, a visually detectable signal can be generated, as well as two individualizing markings 14 and 15 and position markers 16.

In the present case, the test strip unit 11 comprises eight test strips 12 whose width and distances from each other are in accordance with the diameters and distances of a commercially available micro-titer plate with 96 sample tubes. The lower sections of the test strips 12 are, thus, simultaneously insertable into neighboring sample tubes of a tube row of the micro-titer plate. Overall, twelve test strip units can be simultaneously inserted into the different tube rows of the micro-titer plate.

The black arrow indicates the direction in which the liquid is absorbed into the test strips when these are inserted with their lower sections into the sample tubes of a micro-titer plate. The individualizing markings 14 and 15 are provided in the form of bar codes and include manufacture information and/or user-specific information. The position markers 16 enable the image analysis device to localize, and thus, reproducibly detect the test strip unit 11, the test strips 12 and/or the delimited areas 13.

FIG. 2 shows a positioning device 21 with seats 22 for the test strip units 11, with position markers 23, a gray scale 24 and a color scale 25. The positioning device 22 is designed in the form of a surface, graphically recordable by the image generating device. The position markers 23 are graphically recorded by the image generating device so that the image analysis device can localize and thus reproducibly detect the test strip unit 12 by means of the position markers 23. The gray scale 24 and/or the color scale 25 is also graphically recorded by the image generating device and used for calibration by the image analysis device. Depending on the purpose of use, it can be provided that the positioning device 21 only comprises the gray scale 24 or the color scale 25.

In the present case, the positioning device 21 comprises twelve seats 22 for test strip units 11 and can thus accept as many test strip units 11 as are insertable into the different tube rows of a commercially available micro-titer plate with 96 sample tubes.

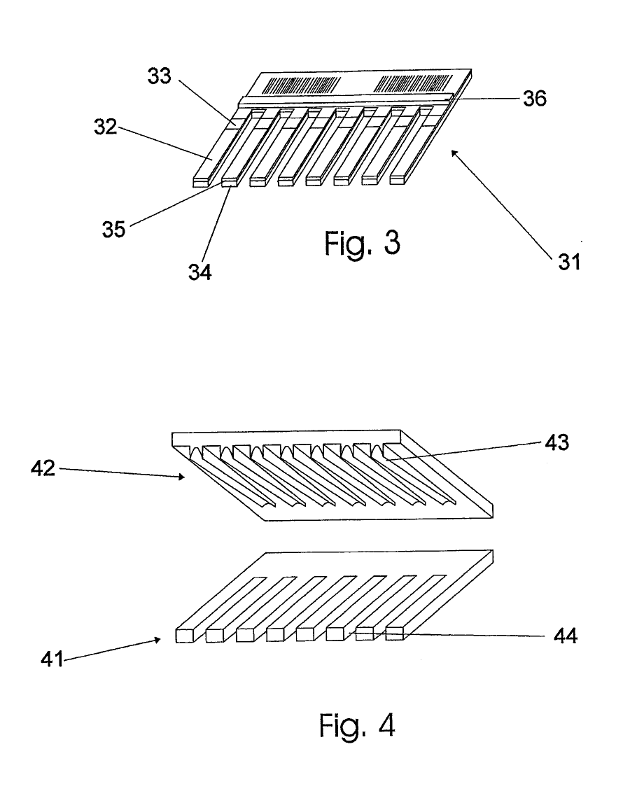

FIG. 3 shows a test strip unit 31 which consists of a plurality of tests strips 32 which are arranged in a defined spatial arrangement to each other, each comprising at least one delimited area 33. In the latter, a visually detectable signal can be generated after contact with a sample to be examined. The test strips 32 consist of an absorbable material 35 which is applied on a stiff carrier material 34. The stiff carrier material can be, for example a plastic, whereas the absorbable material can be, for example, nitrocellulose.

The test strip unit comprises eight tests strips whose diameter and distances from each other are selected such that the lower sections of the individual test strips can be simultaneously inserted into neighboring sample tubes of a tube row of a micro-titer plate.

Moreover, the test strip unit 31 comprises a waste pad which is used for the absorption of any excess liquid. The waste pad can consist of pressed cellulose.

FIG. 4 shows a stamping tool for the production of a test strip unit in which the absorbable cuts of the individual test strips are designed as one piece connected with each other. The stamping tool consists of a stamping plate 41 as well as a stamping tool 42 whose blades 43 have a falling profile. The stamping plate 41 comprises a negative profile in accordance with the shape of the test strip unit to be produced. Prior to the stamping process, a test strip blank is placed onto the stamping plate 41. During the stamping, the falling profiles 43 of the stamping tool 42 engage successively into the cutouts 44 of the negative profile of the stamping plate 41 and, thus, cut out the areas to be separated from the blank.

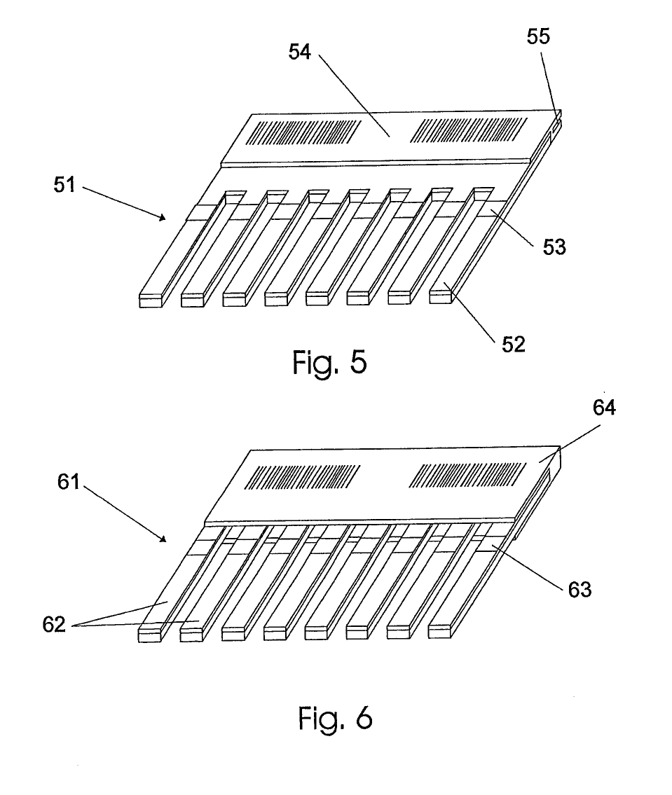

FIG. 5 shows a test strip unit 51 in which the absorbable areas of the individual test strips 52 are designed in one piece connected with each other. The test strip unit comprises, furthermore, an edge reinforcement 54 arranged in an absorbing direction above the delimited areas 53 extending transversely to the test strips 52. In the rear part of the edge reinforcement 54, a groove 55 is provided into which, upon arrangement in the seats of the positioning device, an elastic lip arranged there can engage so that the test strip unit 51 latches into the seat. Especially when the groove is formed on three sides, a particularly secure positioning of the test strip unit can be ensured.

FIG. 6 shows a test strip unit 61 which consists of individual, non-contiguous test strips 62, with the test strips 62 being connected with each other by means of a connecting device 64 arranged in the absorbing direction above the delimited areas 63 extending transversely. Here, the test strips 62 can be glued or tacked into the connecting device 64 or held by it by means of clamping. The connecting device 64 can also act at the same time as an edge reinforcement in the sense already described; in particular, it can also comprise a groove which enables latching into the seats of the positioning device.

1. A device for the visual analysis of test strips (12), each comprising at least one delimited area (13) in which, after contact with a sample to be examined, a visually detectable signal can be generated, with
a) a positioning device (21) comprising at least one seat for a test strip and/or a test strip unit (11 ) consisting of a plurality of test strips (12) connected in a defined spatial arrangement; and
b) an image generating device which graphically records at least one of the delimited areas (13) of a test strip arranged in a seat of the positioning device or of a test strip unit, and which transmits the recording result to an image analysis device,
c) with the image analysis device qualitatively and/or quantitatively analyzing the visually detectable signals for every test strip.

2. A device according to claim 1, wherein the positioning device (21) is designed to be handled separately from the image generating device and to be arranged on the image generating device.

3. A device according to claim 2, characterized in wherein the positioning device (21) and the image generating device are designed such that the positioning device can be reproducibly arranged in a defined arrangement on the image generating device.

4. The device according to claim 1, wherein the image generating device is a scanner.

5. The device according to claim 1, wherein the image analysis device consists of a computer with image processing software.

6. The device according to claim 1, wherein the positioning device (21) consists of a frame, and the seats (22) for the test strip units (11) or the test strips consist of cutouts in the frame (21).

7. The device according to claim 1, wherein the seats (22) are designed for the latching acceptance of the test strip units (11 ) or test strips.

8. The device according to claim 1, wherein the positioning device (21) comprises at least two visually detectable position markers (23).

9. The device according to claim 8, wherein the image generating device graphically records the position markers (23) and transmits the recording result to the image analysis device which can localize and thus reproducibly detect the test strip units (11), the test strips (12) and/or the delimited areas (13) by means of the position markers (23).

10. The device according to claim 1, wherein the test strip units (11) and/or the test strips comprise visually detectable, individualizing markings (14, 15).

11. The device according to claim 10, wherein the individualizing markings (14, 15) are bar codes.

12. The device according to claim 10, wherein the image generating device graphically records the individualizing markings (14, 15) and transmits the recording result to the image analysis device which identifies the individual test strip units (11) and/or the test strips (12) on the basis of the individualizing markings (14, 15), and which uses the data codified in the markings for the analysis.

13. The device according to claim 1, wherein the test strip units (11) and/or the test strips comprise at least two visually detectable position markers (16).

14. The device according to claim 13, wherein the image generating device graphically records the position markers (16) on the test strip units (11) and/or the test strips and transmits the recording result to the image analysis device which can localize and, thus, reproducibly detect the test strip units (11), the test strips (12) and/or the delimited areas (13) by means of the position markers (16).

15. The device according to claim 1, wherein the positioning device (21) comprises a visually detectable gray scale (24) and/or a color scale (25).

16. The device according to claim 15, wherein the image generating device graphically records the gray scale (24) and/or the color scale (25) and transmits the recording result to the image analysis device which uses it for calibration of the analysis of the visually detectable signals on each test strip (12).

17. The device according to claim 1, wherein the test strip unit (11) is designed such that the individual test strips (12) are arranged parallel to each other and spaced apart from each other such that their lower sections can be simultaneously inserted into neighboring sample tubes of a tube row of an integrated sample tube system.

18. The device according to claim 17, wherein the test strip unit (11) is designed such that a plurality of test strip units can be simultaneously inserted with their lower sections into different tube rows of an integrated sample tube system.

19. The device according to claim 1, wherein the image analysis device performs a plausibility check, checking whether
a test strip unit (11) or a test strip is arranged in all seats (22) of the positioning device (21); and/or
whether the individual test strip units (11) or test strips are arranged in a desired sequence in the seats of the positioning device (21); and/or
whether the test strip units (11) or test strips are from the same manufacturing batch; and/or
whether the best before date of the test strip units (11) or of the test strips is already reached.

20. The device according to claim 1, wherein the image analysis device
determines, on the basis of the recording result transmitted by the image generating device, the positions of the test strip units, the test strips and/or the delimited areas;
identifies the individual test strip units, test strips and/or the delimited areas; and
qualitatively and/or quantitatively analyzes visually detectable signals for each test strip.

21. The device according to claim 1, wherein the image analysis device localizes and, thus, reproducibly detects the test strip units (12), the test strips and/or the delimited areas (13) by means of position markers (16, 23) arranged on the positioning device (21), the test strip units (11) and/or the test strips, and graphically recorded by the image generating device.

22. The device according to claim 20, wherein the image analysis device identifies the individual test strip units (11), and/or the individual test strips by means of individualizing markings (14, 15) graphically recorded by the image generating device and arranged on the test strip units (11) and/or the test strips, and which uses the data codified in the markings (14, 15) for the analysis.

23. The device according to claim 1, wherein the image analysis device performs a plausibility check, checking whether
a test strip unit (11) or a test strip is arranged in all seats (22) of the positioning device (21); and/or
whether the individual test strip units (11) or test strips are arranged in a desired sequence in the seats (22) of the positioning device (21); and/or
whether the test strip units (11) or the test strips are from the same manufacturing batch; and/or
whether a best before date of the test strip units (11) or of the test strips is already reached.

24. A positioning device for a device according to claim 1, wherein the positioning device forms a surface graphically recordable by an image generating device and that the positioning device comprises seats for at least one test strip unit or at least one test strip.

25. The positioning device according to claim 24, wherein the positioning device consists of a frame (21), and the seats for the test strip units (11) or the test strips consist of cutouts in the frame (21).

26. The positioning device according to claim 24, wherein the seats (22) are designed for the latching acceptance of the test strip units (11) or test strips.

27. The positioning device according to claim 24, wherein the positioning device (21) comprises at least two visually detectable position markers (23).

28. The positioning device according to claim 24, wherein the positioning device (21) comprises a gray scale (24) and/or a color scale (25).

29. A method for the visual analysis of test strips comprising at least one delimited area (13) in which, after contact with a sample to be examined, a visually detectable signal can be generated, with at least one test strip or at least one test strip unit (11), consisting of a plurality of test strips (12) combined in a defined spatial arrangement, being arranged in a seat (22) of a positioning device (21), which can be arranged on an image generating device, comprising the steps of:
graphically recording at least one of the delimited areas (13) of a test strip arranged in a seat of the positioning device or of a test strip unit, using the image generating device; and
transmitting the recording result to an image analysis device which determines the positions of the individual test strip units (11), test strips (12) and/or the delimited areas (13) and identifies them, and qualitatively and/or quantitatively analyzing the visually detectable signal for each test strip (12).

30. The method according to claim 29, wherein position markers (16, 23), arranged on the positioning device (21), the test strip units (11) and/or the test strips, are graphically recorded by means of the image generating device, and that the recording result will be transmitted to the image analysis device which can localize and thus reproducibly detect the test strip units (11), the test strips, and/or the delimited areas (13) by means of the position markers (16, 23).

31. The method according to claim 29, wherein the image generating device graphically records individualizing markings (14, 15) arranged on the test strip units (11) and/or the test strips, and transmits the recording result to the image analysis device which identifies the individual test strip units (11) or the test strips (12) on the basis of the individualizing markings (14, 15), and which uses the data codified in the markings for the analysis.

32. The method according to claim 30, wherein the image generating device graphically records a gray scale (24) and/or a color scale (25) arranged on the positioning device (21) and transmits the recording result to the image analysis device which uses it as a calibration scale for the analysis of the visually detectable signals on each test strip (12).

33-40. (canceled)

41. A stamping method for the production of a test strip unit comprising the steps of:
placing a blank for a test strip unit (31) which comprises at least the material for an absorbable cut onto a stamping plate (41); and
stamping at least the absorbable cut of the test strip unit with a stamping tool (42), wherein the stamping plate (41) comprises a negative profile corresponding to the cut of the test strip unit (31) and that the blades of the stamping tool (42) used comprise a falling profile (43) which, during the stamping process, successively engages into the cutouts (44) of the negative profile of the stamping plate (41).
